(19) United States
(12) Patent Application Publication
(10) Pub. No.: US 2013/0337056 A1
(43) Pub. Date: Dec. 19, 2013

(54) COATED SOLID PHARMACEUTICAL PREPARATION

(71) Applicants: Lauri Lehtonen, Otzberg/Lengfeld (DE); Heike Bley, Koenigstein im Taunus (DE); Luc Van Der Heyden, Seeheim (DE)

(72) Inventors: Lauri Lehtonen, Otzberg/Lengfeld (DE); Heike Bley, Koenigstein im Taunus (DE); Luc Van Der Heyden, Seeheim (DE)

(73) Assignee: MERCK PATENT GMBH, Darmstadt (DE)

(21) Appl. No.: 14/002,862

(22) PCT Filed: Mar. 1, 2012
(86) PCT No.: PCT/EP12/00883

§ 371 Date: Sep. 3, 2013

(30) Foreign Application Priority Data
Mar. 3, 2011 (EP) 11001791.0

(51) Int. Cl. A61K 9/28

(52) U.S. Cl. CPC A61K 9/2813
USPC 424/451; 424/490; 424/474; 424/93.1; 514/560; 424/93.45

(57) ABSTRACT

The invention is directed to coated solid pharmaceutical preparations having a very thin coating in the nanometer range and a method for producing such preparations. The coated solid pharmaceutical preparation can be prepared by using atomic layer deposition (ALD).

Coatings for pharmaceutical solid preparations are often used in order to mask the flavour or odour of a drug, ensure the safety of the drug by preventing the generation of drug dust, improve the stability of the drug by protecting the drug from light, water and oxygen, and improve the efficacy or stability of the drug by imparting solubility in intestines or controlled release effects.

Methods used for coating of solid pharmaceutical preparations involve e.g. gelatine coating, sugar coating, film coating and powder coating.

Gelatine as coating material for solid pharmaceutical preparations has become less important over the years as it is associated with some drawbacks. Firstly, gelatine is a material obtained from animals which results in a considerable variation of properties between different batches. Secondly, gelatine is in discussion as potential risk factor with regard to inducing bovine spongiform encephalopathy (BSE), and, thirdly, gelatine has an off odours. Furthermore, gelatine is applied as coating in aqueous solution and the presence of water during the coating process and residual moisture in the film may affect stability of certain water sensitive drugs.

Sugar coating has been frequently used in the past but has also become less important due to its several drawbacks. Sugar coating can only be applied to tablets and requires several steps which are time consuming (I. Sealing/Water proofing to provide a moisture barrier and harden the tablet surface, II. Sub coating to cause a rapid buildup and round off the tablet edges, III. Several layering steps to smooth out the subcoated surface and to build up the sugar coat for the increase of the tablet size, IV. Colouring to give the tablet its colour and finished size, V. Smoothing and Polishing). This results in flattening of the tablet shape, disappearance of visibility of engravings and a thick coating, which is subject a higher risk of cracking. Further, sugar coating requires experienced personal, long process times and is difficult to automate.

Film coating is currently the most frequently used coating method. Generally, a mixture of polymers, pigments and excipients is dissolved in an appropriate organic solvent (for water insoluble polymers) or water (for water soluble polymers) to form a solution, or dispersed in water to form a dispersion, and then sprayed onto the dosage forms and dried by continuously providing heat, typically using hot air, until a dry coating film is formed. As organic based film-coating technology suffers toxicological, environmental, cost and safety-related disadvantages aqueous-based coating technology is usually preferred and was developed to phase out organic based coating using water as solvent.

However, aqueous-based coating is associated with other problems such as a slow drying rate of coating, high energy input to remove water, microbial contamination, etc. Furthermore, the presence of water during the coating process and residual moisture in the film may affect stability of certain water sensitive drugs.

As a result both kinds of film coating, the organic and the aqueous film coating techniques involve problems, which are principally associated with solvent used to dissolve or disperse the coating materials. Therefore, a coating process, which does not use an organic solvent or water, seems to be desirable.

Powder coating is an approach to overcome the problems involved with solvents. U.S. Pat. No. 6,117,479 A describes a process of powder coating. In such process electrostatically charged powders are applied to tablets that are fixed in a holder that flips them to expose both sides to the coating. The powder, which adheres to the tablets due to the electrostatic difference, is then fused by applying heat energy (infrared radiation). As the high temperatures necessary to fuse the powder to a coating are detrimental for the active ingredient plasticizers have been added to the coating material to reduce the softening temperature (Ts) or glass transition temperature (Tg) to achieve a feasible operation temperature. However, in order to achieve sufficient coating thickness excessive amount of plasticizers have been found to be necessary, which disadvantageously leads to very soft and sticky films.

WO 2007/014464 A1 discloses that separation of coating step into two steps, where the plasticizer is applied to the tablet in a first step and the further coating material is applied in a subsequent step, lead to some improvement in this respect but does not solve such problem at all.

In general tablets or pellets coated by a powder coating process require higher coating levels/thicker coating layers to obtain similar functional properties (e.g. moisture/oxygen protection or drug release patterns). Powder coating techniques potentially suffer from problems such as the use of high amounts of plasticizers (e.g. Talcum), the need of additional excipients and problems regarding uniformity of film formation dependent on time effective curing processes and ageing problems during storage. Reduction of pinholes and a homogeneous film formation are dependent on the glass transition of the polymers and therefore strongly dependent on process temperature and temperature/humidity conditions during storage. For example products coated with ethylcellulose, which is used very commonly, often exhibits storage problems, especially at climatic zone 4 or 4b, due to its relatively low glass transition temperature (Tg) of about 50/60° C.

The known methods used for coating solid pharmaceutical preparations are associated with several disadvantages as set forth above. Powder coating seems to have some advantages but such technology is rarely used and requires further development. It would be desirable to provide coated pharmaceutical formulations, which don't suffer the problems of the coated formulations of the state-of-the-art as set forth above. In addition, the coated formulations should have a homogeneous pinhole free uniform coating protecting the coated article from external disturbances such as moisture and oxygen and should avoid large layer thicknesses.

The present invention provides coated solid pharmaceutical preparations, which have an ultrathin, conformal coating on the surface thereof. By “ultrathin”, it is meant that the thickness of the coating is up to about 100 nm. By “conformal” it is meant that the thickness of the coating is relatively uniform across the surface of the pharmaceutical preparation, so that the surface shape of the coated pharmaceutical preparation closely resembles that of the uncoated pharmaceutical preparation.

Pharmaceutical preparation above and below is taken to mean a term for various technical administration forms as are known for the administration of medicaments to humans or animals. The expression pharmaceutical preparation is thus independent of a particular legal status and is in no way restricted to medicaments, ingredients which may be present are various substances, such as, for example, medicaments, food supplements and/or functional ingredients. Examples of pharmaceutical preparation for the purposes of the present invention can be in the form of medicaments and food supplements.

According to a preferred embodiment of the invention the coated solid pharmaceutical preparation has a thickness from about 0.1 to about 100 nm, more preferably from about 0.3 to about 50 nm, even more preferably from about 0.5 to about 35 nm and most preferably from about 1 and about 10 nm.

Solid pharmaceutical preparations which are suitable to be converted into the coated solid pharmaceutical preparation include all kinds of solid pharmaceutical preparations such as pellets, granules, tablets or capsules. Accordingly, a preferred embodiment of the invention is characterized in that the solid pharmaceutical preparation is a pellet, a granule, a tablet or a capsule.

A suitable and preferred method for providing such coated pharmaceutical preparations is applying the coating material through atomic layer controlled growth techniques. Therefore, according to a preferred embodiment is directed to a coated solid pharmaceutical preparation, wherein the coating has been applied to the preparation by atomic layer deposition (ALD).

Atomic layer deposition allows the formation of ultrathin coatings by deposition of the coating material as monomolecular layers. Depending from the number of cycles the pharmaceutical preparations can be coated with one or more atomic layers, as described below in more detail. Accordingly, a preferred embodiment of the invention is directed to a coated solid pharmaceutical preparation, wherein the coating comprises one or more atomic layers.

Atomic layer controlled growth techniques permit the deposition of coatings of about 0.1 nm to up to about 0.3 nm in thickness per reaction cycle, and thus provide a means of extremely fine control over coating thickness. In these techniques, the coating is formed in a series of two or more self-limited reactions, which in most instances can be repeated to sequentially deposit additional layers of the coating material until a desired coating thickness is achieved.

According to a preferred embodiment of the invention the coating of the coated solid pharmaceutical preparation has been applied at process temperatures from about 40° C. to about 300° C., more preferably from about 40° C. to about 200° C., even more preferably from about 40° C. to about 150° C. and most preferably from about 50° C. to 100° C.

In most instances, the first of these reactions will involve some functional group on the surface of the pharmaceutical preparation, such as a Z-O—H or Z-N—H group, where Z represents an atom such as a carbon. The individual reactions are advantageously carried out separately and under conditions such that all excess reagents and reaction products are removed before conducting the succeeding reaction.

It is preferred to treat the pharmaceutical preparation before initiating the reaction sequence to remove volatile materials that may be absorbed onto the surface. This is readily done by exposing the pharmaceutical preparation to vacuum. Also, in some instances a precursor reaction may be done to introduce desirable functional groups onto the surface of the pharmaceutical formulation.

In principle all kinds of coatings such as oxide coating, nitride coating or sulfide coating can be applied to the solid pharmaceutical preparation. As pharmaceutical preparations are dedicated to be applied to animals or humans toxicological considerations have to be taken into account in the selection of the coating. From this point of view oxide coatings, especially metal oxide coatings as described hereinafter, are preferred. Accordingly, one preferred embodiment of the invention is directed to a coated solid pharmaceutical preparation, wherein the coating comprises one or more metal oxides.

In one embodiment of the invention each layer of coating is composed of one metal oxide. Accordingly, one preferred embodiment of the invention is directed to a coated solid pharmaceutical preparation, wherein the coating comprises one or more layers, wherein each layer essentially consists of one metal oxide.

Alternatively, the layers of the coating can be also composed of mixtures of two or more metal oxides. Mixtures of different metal oxides in one layer can be used to modify the properties of the layer and to adapt it to the specific demands. Accordingly, another preferred embodiment of the invention is directed to a coated solid pharmaceutical preparation, wherein the coating comprises one or more layers, wherein each layer essentially consists of a mixture of two or more metal oxides.

In principle, if the coating comprises more than one layer each of such layers can be composed of a different metal oxide and/or mixtures of two or more metal oxides. Normally it is preferred that the coating of the solid pharmaceutical preparation has a uniform coating, wherein each of the layers building up such coating consists of the same metal oxide or of the same mixture of two or more metal oxides. Consequently, one further preferred embodiment of the invention is directed to coated solid pharmaceutical preparation, wherein the coating essentially consists of one or more layers, wherein each layer essentially consists of the same metal oxide or of the same mixture of metal oxides.

However, in order to modify its properties, it can be also advantageous that the different layers of the coating are composed of different metal oxides. Variation of the assembly of layers which are composed of different metal oxides and/or mixtures of different metal oxides can be used as a simple tool to adapt the properties of the coating to the different requirements.

Advantageously, the metal/s being present in the coating is/are aluminum, titanium, magnesium, zincum, zirconium and/or silicon, preferably aluminum, titanium and/or zincum Accordingly, the present invention is further directed to a coated solid pharmaceutical preparation, wherein the metal/s, which is/are present in the metal oxide, is/are aluminum, titanium, magnesium, zincum, zirconium and/or silicon, preferably aluminum, titanium, magnesium, zincum, zirconium and/or silicon, preferably aluminum, titanium and/or zincum. More specifically, the present invention is further directed to a coated solid pharmaceutical preparation, wherein the metal oxide/s is/are selected from the group consisting of aluminium oxide (Al2O3), titanium dioxide (TiO2) and magnesium oxide (MgO), zinc oxide (ZnO), zirconium dioxide (ZrO2) and/or silicon dioxide (SiO2), preferably from the group consisting of aluminium oxide (Al2O3), titanium dioxide (TiO2) and zinc oxide (ZnO).

Oxide coatings can be prepared on pharmaceutical preparations having surface hydroxyl (Z-O—H) or amine (Z-N—H) groups using a binary (AB) reaction sequence as follows. The asterisk (*) indicates the atom that resides at the surface of the particle or coating, and Y represents oxygen or nitrogen. M1 is an atom of a metal (or semimetal such as silicon), particularly one having a valence of 2, 3 or 4, and X is a displaceable nucleophilic group. M1 is together with the displaceable nucleophilic group X, which form the reagent M1Xn, is also referred to as precursor.

The reactions shown below are not balanced, and are only intended to show the reactions at the surface of the particles (i.e., not inter- or intralayer reactions).

Z-Y—H*+M1Xn→Z-Y-M1X*+HX (A1)

Z-Y-M1X*+H2O→Z-Y-M1OH*+HX (B1)

In reaction A1, reagent M1 Xn reacts with one or more Z-Y—H* groups on the surface of the pharmaceutical preparation to create a new surface group having the form −M1-X*. M1 is bonded to the pharmaceutical preparation through one or more Y atoms. The −M1-X* group represents a site that can react with water in reaction B1 to regenerate one or more hydroxyl groups. The groups formed in reaction B1 can serve as functional groups through which reactions A1 and B1 can be repeated, each time adding a new layer of M1 atoms. Note that in some cases (such as, e.g., when M1 is silicon, zirconium, titanium, zincum or aluminum) hydroxyl groups can be eliminated as water, forming M1-O-M1 bonds within or between layers. This condensation reaction can be promoted if desired by, for example, annealing at elevated temperatures and/or reduced pressures.

Binary reactions of the general type described by equations A1 and B2, where M1 is aluminum, are described in A. C. Dillon et al, “Surface Chemistry of Al2O3 Deposition using Al(CH3)3 and H2O in a Binary reaction Sequence”, Surface Science 322, 230 (1995) and A. W. Ott et al., “Al2O3 Thin Film Growth on Si(100) Using Binary Reaction Sequence Chemistry”, Thin Solid Films 292, 135 (1997). Both of these references are incorporated herein by reference. General conditions for these reactions as described therein can be adapted to construct SiO2 and Al2O3 coatings on particulate materials in accordance with this invention. Analogous reactions for the deposition of other metal oxides such as ZrO2, TiO2 and B2O3 are described in Tsapatsis et al. (1991) Ind. Eng. Chem. Res. 30:2152-2159 and Lin et al., (1992), AlChE Journal 38:445-454, both incorporated herein by reference.

In the foregoing reaction sequences, suitable metals M1 include silicon, aluminum, titanium, zinc, magnesium and zirconium, whereby aluminum, titanium and magnesium are preferred. Suitable replaceable nucleophilic groups will vary somewhat with M1, but include, for example, fluoride, chloride, bromide, alkoxy, alkyl, acetylacetonate, and the like.

Following ALD as described performance of one cycle results in deposition of one monomolecular layer on the pharmaceutical preparation. If subsequent cycles are performed and the same precursor or different precursors, which contain the same metal, is used in each of this cycles, the whole coating is composed of the same material, which preferably is a metal oxide.

Specific compounds having the structure M1Xn that are of particular interest are silicon tetrachloride (SiCl4), tetramethylorthosilicate (Si(OCH3)4), tetraethyl-orthosilicate (Si(OC2H5)4), trimethyl aluminum (Al(CH3)3), triethyl aluminum (Al(C2H5)3), other trialkyl aluminum compounds, bis(ethylcyclopentadienyl) magnesium (Mg(C2H5C5H4)2), titanium tetraisopropoxide (Ti{OCH(CH3)2}4) and the like.

Specifically preferred are such precursors which allow to conduct the atomic layer deposition at low temperatures up to room temperatures. Such preferred precursors include trimethyl aluminum (Al(CH3)3), bis(ethylcyclopentadienyl) magnesium (Mg(C2H5C5H4)2) and titanium tetraisopropoxide (Ti{OCH(CH3)2}4), titanium tetrachloride (TiCl4) or diethyl zinc (Zn(C2H5)2). Therefore, according to a preferred embodiment of the invention the precursor/s is/are a titanium precursor such as trimethyl aluminum (Al(CH3)3), a magnesium precursor such as bis(ethylcyclopentadienyl) magnesium (Mg(C2H5C5H4)2), and/or a titanium precursor such as titanium tetraisopropoxide (Ti{OCH(CH3)2}4) and titanium tetrachloride (TiCl4) or diethyl zinc (Zn(C2H5)2).

The invention is also directed to a method for producing the coated solid pharmaceutical preparation as described herein, characterized in that the following steps are conducted (a) introducing into a reactor pre-filled with the solid pharmaceutical preparation to be coated a first precursor, which is in a gaseous state, (b) purging and/or evacuating the reactor to remove the non-reacted precursors and the gaseous reaction by-products (c) exposing of the second precursor—to activate the surface again for the reaction of the first precursor (d) purging and/or evacuating of the reactor and optionally repeating the steps (a) to (d) in order to achieve the desired coating thickness.

A convenient method for applying the ultrathin, conformal coating to the base is to form a fluidized bed of the solid pharmaceutical preparations, and then pass the various reagents in turn through the fluidized bed under reaction conditions. Methods of fluidizing solid pharmaceutical preparations are well known, and generally include supporting the solid pharmaceutical preparations on a porous plate or screen. A fluidizing gas is passed upwardly through the plate or screen, lifting the solid pharmaceutical preparations somewhat and expanding the volume of the bed. With appropriate expansion, the solid pharmaceutical preparations behave much as a fluid. Fluid (gaseous or liquid) reagents can be introduced into the bed for reaction with the surface of the solid pharmaceutical preparations.

In this invention, the fluidizing gas also can act as an inert purge gas for removing unreacted reagents and volatile or gaseous reaction products. In addition, the reactions can be conducted in a rotating cylindrical vessel or a rotating tube.

If desired, multiple layers of ultrathin coatings can be deposited on the solid pharmaceutical preparations. This method is of specific interest where, due to the chemical nature of the base solid pharmaceutical preparation, the desired coating cannot easily be applied directly to the particle surface. In such cases, an intermediate ultrathin layer can be applied to provide a surface to which the desired outer layer can be applied more easily.

Another advantage is that the invention will minimize the level of coating material used, as compared to existing film coating techniques. The invention only requires a very thin layer, thereby needing only a minimum amount of material for the coating to be effective against water and oxygen penetration. Minimizing of the amount of coating material is especially desired if a coating material is used, which shouldn't be taken in in large quantities, such as aluminium oxide. Furthermore, if such materials are used the quantity of them can be further reduced by mixing the them with other metal oxides, such as titanium oxide.

Coating of vitamin c tablets with TiO2, Al2O3 and a mixture of TiO2+Al2O3 shows significant variation in the solubility rates, where pure Al2O3 coated tablet has the fastest solubility rate and TiO2 the lowest. The blend of the two mineral oxide coatings has a solubility between the two pure metal oxides. This allows the control of the solubility of the tablet by altering the proportions of the different metal oxides in the layer.

The examples explain the invention without being restricted thereto.

### EXAMPLES

### 1. Example

A probiotic strain containing multilayer tablet weighing around 1000-1200 mg is compressed and coated with either
aluminium oxide (Al2O3) or
titanium dioxide (TiO2) or
zinc oxide (ZnO) or
a mixture of aluminium oxide (Al2O3) and titanium dioxide (TiO2), wherein the coating has a thickness of about 5 to about 40 nm, preferable 10 nm. The processing temperature range is from 40° C. to 70° C., preferable 50° C. The atomic layer deposited coated probiotic mulitilayer tablets packed in a polypropylene bottle are stored at different temperatures and humidity conditions (25° C./60% r.H and 40° C./75% r.H.) to measure the probiotic count over the storage time of 3 months. In order to compare the effect of the ALD coating on the probiotical counts also the multilayer tablets without an ALD coating packed in a polypropylene bottle are investigated.

### 2. Example

A probiotic strain containing multilayer film coated tablet weighing around 1000-1200 mg is compressed, coated with and organic/aqueous HPMC/HPC coating and finally coated with either
aluminium oxide (Al2O3) or
titanium dioxide (TiO2) or
zinc oxide (ZnO) or,
a mixture of aluminium oxide (Al2O3) and titanium dioxide (TiO2), wherein the coating has a thickness of about 5 to about 40 nm, preferable 10 nm. The processing temperature range is from 40° C. to 70° C., preferable 50° C. The atomic layer deposited coated probiotic multilayer film coated tablets packed in a polypropylene bottle are stored at different temperatures and humidity conditions (25° C./60% r.H and 40° C./75% r.H.) to measure the probiotic count over the storage time of 3 months. In order to compare the effect of the ALD coating on the probiotical counts also the multilayer film coated tablets without an ALD coating packed in a polypropylene bottle are investigated.

### 3. Example

A fish oil containing soft gel capsule is coated with either
aluminium oxide (Al2O3) or
titanium dioxide (TiO2) or
zinc oxide (ZnO)
a mixture of aluminium oxide (Al2O3) and titanium dioxide (TiO2), wherein the coating has a thickness of about 5 to about 40 nm, preferable 10 nm. The processing temperature range is from 40° C. to 70° C., preferable 50° C. The atomic layer deposited fish oil soft gel capsules packed in a polypropylene bottle are stored at different temperatures and humidity conditions (25°/60% r.H and 40° C./75% r.H.) to measure the peroxide value over the storage time of 3 months. In order to compare the effect of the coating on the oxidation ratio also fish oil soft gel capsules without any coating packed in a polypropylene bottle are investigated. The atomic layer deposited coated fish oil soft gel capsules are compared to fish oil soft gel capsules without any coating relating their improved sensory properties as taste and smell. ALD coating leads to a reduction of fishy taste and smell.

### 4. ALD Process

ALD process was run using an ALD tool. Before start of the process one of each pharmaceutical preparations tablet or capsule were placed in a can which was placed in an ALD chamber for testing the vacuum and temperature tolerance. No changes in colour or performance of tablets or capsules were observed.

The tablets/capsules were loaded on shelves in a cassette, layer thickness was monitored using Si-monitors. The process was run using the precursors trimethyl aluminum (TMA), titanium tetrachloride (TiCl4), diethyl zinc (DEZ) leading to aluminium oxide (Al2O3), titanium dioxide (TiO2) and zinc oxide (ZnO) coatings.

For coating the tablets/capsules were preheated to the process temperature and coated by consecutively pulsing with the respective precursor, purging with nitrogen, pulsing with water or ozone (O3) and purging with nitrogen. Such procedure was repeated until the desired layer thickness was obtained. The process parameters used for the different coatings are summarized in table 1.

TABLE 1

| | Process | | Thick- | |
|---|---|---|---|---|
| Process | temp. | Coating | ness | Precursor (pulse s/purge s)* + |
| No. | [° C.] | material | [nm] | H2O (pulse s/purge s)* |
| 1 | 60 | Al2O3 | 10 | TMA (1.0/33.0) + H2O (1.5/56.0) |
| 2 | 50 | TiO2 | 10 | TiCl4 (1.5/33.0) + H2O (1.5/56.0) |
| 3 | 50 | Al2O3/ | 10/10 | TMA (2.0/33.0) + H2O (2.0/60.0)/ |
| | | TiO2 | | TiCl4 (2.0/33.0 + H2O 2.0/60.0) |
| 4 | 50 | ZnO | 10 | DEZ (2.0/33.0 + H2O 2.0/60.0) |

*)pulse s/purge s: duration of pulse (with precursor or H2O) in seconds/duration of purge (with nitrogen) in seconds

The following pharmaceutical dosage forms were coated using the process described in table 1:

A) Multilayer tablet

The 3-layer tablet contains several vitamins in a first layer, several minerals and trace elements in a second layer and probiotic microorganisms in a third layer. 3-layer tablet did not contain any coating.

B) Multilayer film coated tablet

The 3-layer tablet is the same as described the multilayer tablet but differs from it in that it is film-coated with an organic/aqueous HPMC/HPC coating layer.

C) Fish oil capsules

Fish oil capsules are oblong shaped, transparent gelatine soft gel capsules containing 1105 mg fish oil concentrate (EPA 33%, DHA 22%, Vitamin E).

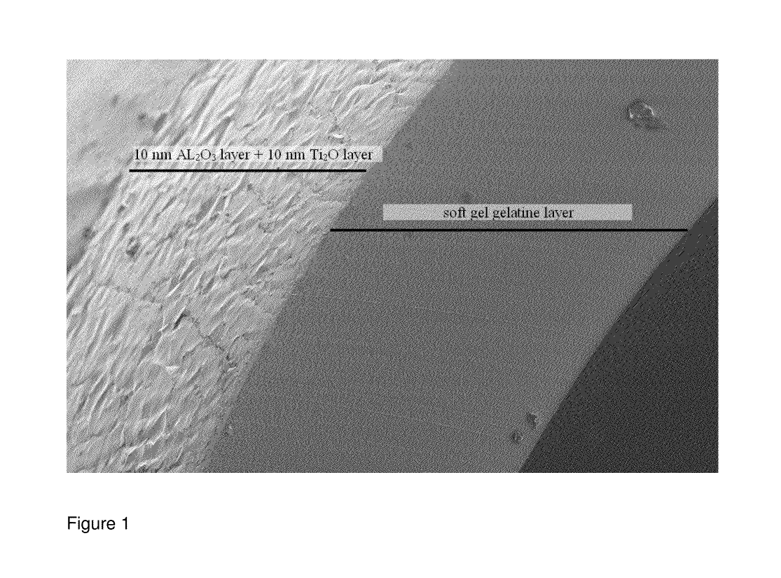

ALD coating process on the pharmaceutical dosage forms led to coated pharmaceutical dosage forms, which comply with the specification of such dosage forms (uniformity of mass, disintegration, hardness). The good quality of the coating was further acknowledged by electron microscopic photography (see FIG. 1 showing a sectional view of fish oil capsules coated by ALD process number 3)

To examine the effect of ALD coating on the stability of the dosage forms the pharmaceutical preparations with and without ALD coating were packed into polypropylene (PP) containers, closed with PP caps and stored at 25° C. and 60% relative humidity (25° C./60% r.h) as well as at 40° C. and 75% relative humidity (40° C./75% r.h.).

Chemical stability of the 3-layer tablets, 3-layer film coated tablets and fish oil capsules was tested directly after manufacture (start) as well as after 3 months storage under the conditions described before. In the 3-layer tablets/film coated tablets vitamin C content and the amounts of probiotic microorganisms were determined, in the fish oil capsules the iodine, peroxide values and anisidine were tested.
For the determination of the Vitamin C assay the tablet/tablet layer with the vitamin C is titrated with 0.5% Chloramin T as standard solution.
The amounts of probiotic microorganisms are tested in a microbiological laboratory by dissolving the tablets in a buffer solution. Agar plates are incubated with the diluted samples at 36° C. and number of viable cells are counted after 48-72 h.
The iodine value is tested by titrating the fat together with calomel (Hg2Cl2) using iodine as standard solution.
The peroxide value is tested in an iodine-starch reaction.
To test the anisidine value the samples are solved in Isooctan/glacial acetic acid and after several minutes the extinction is analysed.

The iodine value is a measure of the unsaturation of fats and oils and is expressed in terms of the number of centrigrams of iodine absorbed per gram of sample (% iodine absorbed). The peroxide value is defined as the amount of peroxide oxygen per 1 kilogram of oil and indicates the degree to which a fat has been oxidized. The anisidine value is defined as the optical density measured at 350 nm, multiplied by 100 of the solution of 1 gram of oil in 100 mL of p-anisidine and is a measure used to assess the secondary oxidation of oil or fat, which is mainly imputable to aldehydes and ketones, and is therefore able to tell the oxidation “history” of the oil.

The test results of the 3-layer tablets are presented in table 2, the test results of the film coated 3-layer tablets are presented in table 3 and the test results of the fish oil capsules are presented in table 4.

TABLE 2

| | | 13 weeks | 13 weeks |
|---|---|---|---|
| Test | Start | 25° C./60% r.h. | 40° C./75% r.h |
| | 3-layer tablet | | |
| Viable cells | 9.0 × 107 | 1.3 × 108 | 1.4 × 106 |
| Lactobacillus gasseri | | | |
| [CFU/g] | | | |
| Viable cells | 1.2 × 107 | 7.3 × 106 | <10 |
| Bifidobacterium | | | |
| bifidum | | | |
| Bifidobacterium | | | |
| longum | | | |
| [CFU/g] | | | |
| Vitamin C [mg] | 70.4 | 71.0 | 70.1 |
| | 3-layer tablet coated by process no. 1 | | |
| Viable cells | 1.6 × 108 | 4.1 × 107 | <100 |
| Lactobacillus gasseri | | | |
| [CFU/g] | | | |
| Viable cells | 9.1 × 106 | 3.2 × 103 | <10 |
| Bifidobacterium | | | |
| bifidum | | | |
| Bifidobacterium | | | |
| longum [CFU/g] | | | |
| Vitamin C [mg] | 70.5 | 71.4 | 69.3 |
| | 3-layer tablet coated by process no. 2 | | |
| Viable cells | 3.9 × 108 | 1.6 × 108 | 9.4 × 107 |
| Lactobacillus gasseri | | | |
| [CFU/g] | | | |
| Viable cells | 2.2 × 107 | 1.4 × 107 | 1.2 × 106 |
| Bifidobacterium | | | |
| bifidum | | | |
| Bifidobacterium | | | |
| longum [CFU/g] | | | |
| Vitamin C [mg] | 72.4 | 71.9 | 71.3 |
| | 3-layer tablet coated by process no. 3 | | |
| Viable cells | 3.5 × 108 | 1.5 × 108 | 8.0 × 107 |
| Lactobacillus gasseri | | | |
| [CFU/g] | | | |
| Viable cells | 4.3 × 107 | 1.1 × 107 | 1.8 × 106 |
| Bifidobacterium | | | |
| bifidum | | | |
| Bifidobacterium | | | |
| longum | | | |
| [CFU/g] | | | |
| Vitamin C [mg] | 71.8 | 72.2 | 70.7 |
| | 3-layer tablet coated by process no. 4 | | |
| Viable cells | 1.2 × 108 | 4.5 × 107 | 1.6 × 104 |
| Lactobacillus gasseri | | | |
| [CFU/g] | | | |
| Viable cells | 1.2 × 107 | 2.1 × 106 | <10 |
| Bifidobacterium | | | |
| bifidum | | | |
| Bifidobacterium | | | |
| longum | | | |
| [CFU/g] | | | |
| Vitamin C [mg] | 71.8 | 72.2 | 70.0 |

As clearly shown by table 2 ALD coating leads to an improvement of storage stability of viable cells whereby the stability of vitamin C is not influenced.

TABLE 3

| | | 13 weeks | 13 weeks |
|---|---|---|---|
| Test | Start | 25° C./60% r.h. | 40° C./75% r.h |
| | film-coated 3-layer tablet | | |
| Viable cells | 6.4 × 107 | 1.7 × 108 | <100 |
| Lactobacillus gasseri | | | |
| [CFU/g] | | | |
| Viable cells | 1.0 × 107 | 1.3 × 107 | <10 |
| Bifidobacterium | | | |
| bifidum | | | |
| Bifidobacterium | | | |
| longum | | | |
| [CFU/g] | | | |
| Vitamin C [mg] | 68.9 | 69.4 | 69.9 |
| | film-coated 3-layer tablet further coated by | | |
| | process no. 1 | | |
| Viable cells | 5.7 × 108 | 1.1 × 108 | 4.0 × 107 |
| Lactobacillus gasseri | | | |
| [CFU/g] | | | |
| Viable cells | 3.9 × 107 | 5.3 × 106 | 1.3 × 106 |
| Bifidobacterium | | | |
| bifidum | | | |
| Bifidobacterium | | | |
| longum [CFU/g] | | | |
| Vitamin C [mg] | 70.1 | 70.6 | 70.7 |
| | film-coated 3-layer tablet further coated by | | |
| | process no. 2 | | |
| Viable cells | 3.7 × 108 | 1.2 × 108 | 2.2 × 107 |
| Lactobacillus gasseri | | | |
| [CFU/g] | | | |
| Viable cells | 4.1 × 107 | 1.0 × 107 | 30 |
| Bifidobacterium | | | |
| bifidum | | | |
| Bifidobacterium | | | |
| longum [CFU/g] | | | |
| Vitamin C [mg] | 70.1 | 70.8 | 71.7 |
| | film-coated 3-layer tablet further coated by | | |
| | process no. 3 | | |
| Viable cells | 5.4 × 108 | 1.6 × 108 | 4.1 × 107 |
| Lactobacillus gasseri | | | |
| [CFU/g] | | | |
| Viable cells | 5.5 × 107 | 9.6 × 106 | 5.7 × 103 |
| Bifidobacterium | | | |
| bifidum | | | |
| Bifidobacterium | | | |
| longum | | | |
| [CFU/g] | | | |
| Vitamin C [mg] | 70.8 | 70.6 | 71.0 |
| | film-coated 3-layer tablet further coated by | | |
| | process no. 4 | | |
| Viable cells | 1.4 × 108 | 5.0 × 107 | 1.7 × 105 |
| Lactobacillus gasseri | | | |
| [CFU/g] | | | |
| Viable cells | 9.2 × 106 | 2.4 × 106 | <10 |
| Bifidobacterium | | | |
| bifidum | | | |
| Bifidobacterium | | | |
| longum | | | |
| [CFU/g] | | | |
| Vitamin C [mg] | 69.2 | 69.4 | 70.4 |

As clearly shown by table 3 ALD coating leads to an improvement of storage stability of viable cells whereby it does not has a detrimental effect on the stability of vitamin C. Such stabilization effect occurs although the initial formulation was already film-coated and, therefore, indicates a stabilization effect in addition to such film coating.

TABLE 4

| | | 13 weeks | 13 weeks |
|---|---|---|---|
| Test | Start | 25° C./60% r.h. | 40° C./75% r.h |
| | Fish oil capsule | | |
| iodine value | 266 | 258 | 266 |
| peroxide value | 1.0 | 4.6 | 6.7 |
| [m · eq./kg O2] | | | |
| anisidine value | 7.19 | 11.16 | 13.76 |
| | Fish oil capsule coated by process no. 1 | | |
| iodine value | 262 | 258 | 270 |
| peroxide value | 0.3 | 5.1 | 4.3 |
| [m · eq./kg O2] | | | |
| anisidine value | 10.57 | 10.85 | 11.44 |
| | Fish oil capsule coated by process no. 2 | | |
| iodine value | 262 | 257 | 264 |
| peroxide value | 0.3 | 2.5 | 4.4 |
| [m · eq./kg O2] | | | |
| anisidine value | 10.09 | 10.78 | 11.58 |
| | Fish oil capsule coated by process no. 3 | | |
| iodine value | 262 | n.d. | n.d. |
| peroxide value | 0.3 | n.d. | n.d. |
| [m · eq./kg O2] | | | |
| anisidine value | 10.89 | n.d. | n.d. |
| | Fish oil capsule coated by process no. 4 | | |
| iodine value | 270 | 270 | 271 |
| peroxide value | 6.5 | 4.0 | 4.49 |
| [m · eq./kg O2] | | | |
| anisidine value | 10.47 | 8.99 | 11.75 |

As clearly shown by table 4, ALD coating leads to significantly decreased peroxide values. Further, the iodine and anisidine values for the ALD coated capsules are at least as good as the capsules without ALD coating. Therefore overall stability of fish oil capsules is significantly increased by ALD coating.

1. Coated solid pharmaceutical preparation comprising at least one active ingredient, wherein the coating has a thickness of from about 0.1 to about 100 nm, preferably from about 0.3 to about 50 nm, more preferably from about 0.5 to about 35 nm.

2. Coated solid pharmaceutical preparation according to claim 1, wherein the pharmaceutical preparation is a pellet, a granule, a tablet or a capsule.

3. Coated solid pharmaceutical preparation according to claim 1, wherein the coating has been applied to the preparation by atomic layer deposition (ALD).

4. Coated solid pharmaceutical preparation according to claim 3, wherein the coating comprises one or more atomic layers.

5. Coated solid pharmaceutical preparation according to claim 1, wherein the coating comprises one or more metal oxides.

6. Coated solid pharmaceutical preparation according to claim 5, wherein the coating comprises one or more layers, wherein each layer essentially consists of one metal oxide.

7. Coated solid pharmaceutical preparation according to claim 6, wherein the coating essentially consists of one or more layers, wherein each layer essentially consists of one metal oxide.

8. Coated solid pharmaceutical preparation according to claim 5, wherein the coating comprises one or more layers, wherein each layer essentially consists of a mixture of two or more metal oxides.

9. Coated solid pharmaceutical preparation according to claim 5, wherein the coating essentially consists of one or more layers, wherein each layer essentially consists of the same metal oxide or of the same mixture of metal oxides.

10. Coated solid pharmaceutical preparation according to claim 5, wherein the metal/s, which is/are present in the metal oxide, is/are aluminum, titanium, magnesium, zincum, zirconium and/or silicon, preferably aluminum, titanium, zincum and/or magnesium.

11. Coated solid pharmaceutical preparation according to claim 10, wherein the metal oxide/s is/are selected from the group consisting of aluminium oxide (Al₂O₃), titanium dioxide (TiO₂) and magnesium oxide (MgO), zinc oxide (ZnO), zirconium dioxide (ZrO₂) and/or silicon dioxide (SiO₂), preferably from the group consisting of aluminium oxide (Al₂O₃), titanium dioxide (TiO₂), zincum oxide (ZnO) and magnesium oxide (MgO).

12. A method for producing the coated solid pharmaceutical preparation according to claim 1, characterized in that the following steps are conducted (a) introducing into a reactor pre-filled with the solid pharmaceutical preparation to be coated a first precursor, which is in a gaseous state, (b) purging and/or evacuating the reactor to remove the non-reacted precursors and the gaseous reaction by-products (c) exposing of the second precursor—to activate the surface again for the reaction of the first precursor (d) purging and/or evacuating of the reactor and optionally repeating the steps (a) to (d) in order to achieve the desired coating thickness.

13. The method according to claim 12, wherein the precursor/s is/are a titanium precursor such as trimethyl aluminum (Al(CH₃)₃), a magnesium precursor such as bis(ethylcyclopentadienyl) magnesium (Mg(C₂H₅C₅H₄)₂), and/or a titanium precursor such as titanium tetraisopropoxide (Ti{OCH(CH₃)₂}₄) and titanium tetrachloride (TiCl₄) or diethyl zinc (Zn(C₂H₅)₂).

14. The method according to claim 12, wherein the second precursor is an oxidant such as water, hydrogen peroxide and/or ozone, preferably water.
